(19) United States
(12) Patent Application Publication
(10) Pub. No.: US 2026/0240505 A1
(43) Pub. Date: Aug. 20, 2026

(54) EAR-WEARABLE SYSTEMS FOR OVERPRESSURE DETECTION AND EFFECT MONITORING

(71) Applicant: Starkey Laboratories, Inc., Eden Prairie, MN (US)

(72) Inventor: David Alan Fabry, Eden Prairie, MN (US)

(21) Appl. No.: 19/181,550

(22) Filed: Apr. 17, 2025

Related U.S. Application Data
(60) Provisional application No. 63/635,393, filed on Apr. 17, 2024.

(51) Int. Cl. A61B 5/00; A61B 5/021; A61B 5/11

(52) U.S. Cl. CPC A61B 5/7282; A61B 5/021; A61B 5/112; A61B 5/4023; A61B 5/4806

(57) ABSTRACT

Embodiments herein relate to ear-wearable devices and systems that can detect overpressure events. Embodiments herein relate to ear-wearable devices and systems that can monitor individuals for the effects of exposure to overpressure events. In an embodiment, an ear-wearable system is included having a control circuit and a sensor package in electrical communication with the control circuit. The sensor package can include a motion sensor and one or more sound input devices. The ear-wearable system can be configured to detect an overpressure event using the sensor package and monitor physiological effects of the overpressure event. Other embodiments are also included herein.

This application claims the benefit of U.S. Provisional Application No. 63/635,393, filed Apr. 17, 2024, the content of which is incorporated herein by reference in its entirety.

### FIELD

Embodiments herein relate to ear-wearable devices and systems that can detect overpressure events. Embodiments herein relate to ear-wearable devices and systems that can monitor individuals for the effects of exposure to overpressure events.

### BACKGROUND

Explosive blasts or explosions are physical phenomena that result in a sudden release of energy. This process causes a near instantaneous compression of the surrounding medium (e.g., air or water) and an increase in pressure (“overpressure”) above atmospheric pressure (“an overpressure event”), resulting in an overpressure wave (or blast wave). This overpressure wave propagates outward from the explosion in a radial fashion at supersonic speed, creating a wake of negative pressure (“underpressure”) that follows behind. Overpressure events can occur in the context of military activities, industrial scenarios, certain sports (such as motorsports and shooting sports), certain research activities, and the like.

Overpressure events can cause significant damage to structures and injury to humans and other animals. In general, the severity of these injuries depends on the proximity to the blast and the magnitude of the overpressure, with higher pressures increasing the likelihood of severe trauma or fatality. Overpressure injuries can be divided into four classes: primary, secondary, tertiary, and quaternary. Primary injuries are those caused directly by the blast overpressure wave. Primary injuries can affect air-filled organs like the ears and lungs, potentially leading to eardrum rupture or lung damage. Secondary injuries result from flying debris and shrapnel. Tertiary injuries occur when individuals are physically thrown by the blast wind, leading to blunt trauma, fractures, or traumatic amputations. Quaternary injuries can encompass a variety of other injuries, including, for example, burns, radiation exposure, and inhalation of toxic gases.

### SUMMARY

Embodiments herein relate to ear-wearable devices and systems that can detect overpressure events. Embodiments herein also relate to ear-wearable devices and systems that can monitor individuals for the effects of exposure to overpressure events.

In a first aspect, an ear-wearable system can be included having a control circuit, and a sensor package in electrical communication with the control circuit. The sensor package can include a motion sensor, and one or more sound input devices. The ear-wearable system can be configured to detect an overpressure event using the sensor package and monitor physiological effects of the overpressure event.

In a second aspect, in addition to one or more of the preceding or following aspects, or in the alternative to some aspects, the physiological effects can include vestibular system effects.

In a third aspect, in addition to one or more of the preceding or following aspects, or in the alternative to some aspects, the physiological effects can include at least one selected from the group consisting of a change in balance, a change in gait, a change in posture, a change in blood pressure, and a change in heart rate.

In a fourth aspect, in addition to one or more of the preceding or following aspects, or in the alternative to some aspects, the physiological effects can include cognitive performance effects.

In a fifth aspect, in addition to one or more of the preceding or following aspects, or in the alternative to some aspects, the physiological effects can include speech changes.

In a sixth aspect, in addition to one or more of the preceding or following aspects, or in the alternative to some aspects, the physiological effects can include sleep disturbances.

In a seventh aspect, in addition to one or more of the preceding or following aspects, or in the alternative to some aspects, the ear-wearable system can be configured to monitor the physiological effects by comparing baseline values versus after overpressure event values.

In an eighth aspect, in addition to one or more of the preceding or following aspects, or in the alternative to some aspects, the ear-wearable system can be configured to monitor the physiological effects by comparing baseline values versus after overpressure event values for one or more of balance, gait, posture, blood pressure, heart rate and blood perfusion.

In a ninth aspect, in addition to one or more of the preceding or following aspects, or in the alternative to some aspects, the overpressure event can include a blast wave.

In a tenth aspect, in addition to one or more of the preceding or following aspects, or in the alternative to some aspects, the overpressure event can include a peak overpressure of greater than 5 psi.

In an eleventh aspect, in addition to one or more of the preceding or following aspects, or in the alternative to some aspects, the overpressure event can include a peak overpressure of greater than 15 psi.

In a twelfth aspect, in addition to one or more of the preceding or following aspects, or in the alternative to some aspects, the ear-wearable system can be configured to determine a direction of the overpressure event source relative to a system wearer.

In a thirteenth aspect, in addition to one or more of the preceding or following aspects, or in the alternative to some aspects, the ear-wearable system can be configured to determine a direction of the overpressure event source relative to multiple system wearers.

In a fourteenth aspect, in addition to one or more of the preceding or following aspects, or in the alternative to some aspects, the ear-wearable system can be configured to determine a magnitude of the overpressure event.

In a fifteenth aspect, in addition to one or more of the preceding or following aspects, or in the alternative to some aspects, the ear-wearable system can be configured to estimate a magnitude of the overpressure event that saturates one or more sensors of the sensor package.

In a sixteenth aspect, in addition to one or more of the preceding or following aspects, or in the alternative to some aspects, the ear-wearable system can be configured to calculate a maximum rate of change from a starting ambient pressure to a peak pressure of the overpressure event.

In a seventeenth aspect, in addition to one or more of the preceding or following aspects, or in the alternative to some aspects, the ear-wearable system can be configured to calculate a positive duration time of the overpressure event.

In an eighteenth aspect, in addition to one or more of the preceding or following aspects, or in the alternative to some aspects, the ear-wearable system can be configured to calculate a negative duration time of the overpressure event.

In a nineteenth aspect, in addition to one or more of the preceding or following aspects, or in the alternative to some aspects, the ear-wearable system can be configured to classify a severity of the physiological effects of the overpressure event.

In a twentieth aspect, in addition to one or more of the preceding or following aspects, or in the alternative to some aspects, the ear-wearable system can be configured to receive data from other ear-wearable systems regarding the overpressure event.

In a twenty-first aspect, in addition to one or more of the preceding or following aspects, or in the alternative to some aspects, the one or more sound input devices includes a microphone.

In a twenty-second aspect, in addition to one or more of the preceding or following aspects, or in the alternative to some aspects, the one or more sound input devices includes an inward facing microphone.

In a twenty-third aspect, in addition to one or more of the preceding or following aspects, or in the alternative to some aspects, the inward facing microphone can be used to measure a time to reflection of sound within the ear.

In a twenty-fourth aspect, in addition to one or more of the preceding or following aspects, or in the alternative to some aspects, the ear-wearable system can be configured to compare a time to reflection of sound within the ear before and after the overpressure event.

In a twenty-fifth aspect, in addition to one or more of the preceding or following aspects, or in the alternative to some aspects, the sensor package the ear-wearable system can further include an outward facing microphone, and an inward facing microphone, and wherein the ear-wearable system can be configured to compare magnitudes of signals from the outward facing microphone with magnitudes of signals from the inward facing microphone.

In a twenty-sixth aspect, in addition to one or more of the preceding or following aspects, or in the alternative to some aspects, the ear-wearable system can further include a first ear-wearable device and a second ear-wearable device, wherein each of the first ear-wearable device and the second ear-wearable device can have a separate control circuit and sensor package.

In a twenty-seventh aspect, in addition to one or more of the preceding or following aspects, or in the alternative to some aspects, wherein data from sensors of both the first ear-wearable device and the second ear-wearable device can be used to detect and/or analyze the overpressure event.

In a twenty-eighth aspect, in addition to one or more of the preceding or following aspects, or in the alternative to some aspects, the ear-wearable system can be configured to use head shadow and time of arrival effects within signals from the first ear-wearable device relative to signals from the second ear-wearable device to characterize a direction and/or magnitude of the overpressure event.

In a twenty-ninth aspect, in addition to one or more of the preceding or following aspects, or in the alternative to some aspects, the ear-wearable system can be configured to measure head movement associated with the overpressure event.

In a thirtieth aspect, in addition to one or more of the preceding or following aspects, or in the alternative to some aspects, the sensor package can further include an infrared sensor.

In a thirty-first aspect, in addition to one or more of the preceding or following aspects, or in the alternative to some aspects, the one or more sound input devices include a high saturation limit MEMS or electret microphone.

In a thirty-second aspect, in addition to one or more of the preceding or following aspects, or in the alternative to some aspects, the ear-wearable system can be configured to track data associated with multiple overpressure events over time.

In a thirty-third aspect, in addition to one or more of the preceding or following aspects, or in the alternative to some aspects, the ear-wearable system can be configured to query a system wearer after detection of the overpressure event.

In a thirty-fourth aspect, in addition to one or more of the preceding or following aspects, or in the alternative to some aspects, the ear-wearable system can be configured to send data to a remote system after detection of the overpressure event.

In a thirty-fifth aspect, in addition to one or more of the preceding or following aspects, or in the alternative to some aspects, can further include a speaker or receiver.

In a thirty-sixth aspect, a method of evaluating overpressure event injury can be included. The method can include detecting an overpressure event using one or more sensors of a sensor package of an ear-wearable device and monitoring physiological effects of the overpressure event using one or more sensors of the sensor package.

In a thirty-seventh aspect, in addition to one or more of the preceding or following aspects, or in the alternative to some aspects, the method can further include monitoring the physiological effects by comparing baseline values versus after overpressure event values.

In a thirty-eighth aspect, in addition to one or more of the preceding or following aspects, or in the alternative to some aspects, the method can further include monitoring the physiological effects by comparing baseline values versus after overpressure event values for one or more of balance, gait, posture, blood pressure, heart rate and blood perfusion.

In a thirty-ninth aspect, in addition to one or more of the preceding or following aspects, or in the alternative to some aspects, the method can further include determining a direction of the overpressure event source relative to a system wearer.

In a fortieth aspect, in addition to one or more of the preceding or following aspects, or in the alternative to some aspects, the method can further include determining a direction of the overpressure event source relative to multiple system wearers.

In a forty-first aspect, in addition to one or more of the preceding or following aspects, or in the alternative to some aspects, the method can further include determining a magnitude of the overpressure event.

In a forty-second aspect, in addition to one or more of the preceding or following aspects, or in the alternative to some aspects, the method can further include estimating a magnitude of the overpressure event that saturates one or more sensors of the sensor package.

In a forty-third aspect, in addition to one or more of the preceding or following aspects, or in the alternative to some aspects, the method can further include calculating a maximum rate of change from a starting ambient pressure to a peak pressure of the overpressure event.

In a forty-fourth aspect, in addition to one or more of the preceding or following aspects, or in the alternative to some aspects, the method can further include calculating a positive duration time of the overpressure event.

In a forty-fifth aspect, in addition to one or more of the preceding or following aspects, or in the alternative to some aspects, the method can further include calculating a negative duration time of the overpressure event.

In a forty-sixth aspect, in addition to one or more of the preceding or following aspects, or in the alternative to some aspects, the method can further include classifying a severity of the physiological effects of the overpressure event.

In a forty-seventh aspect, in addition to one or more of the preceding or following aspects, or in the alternative to some aspects, the method can further include receiving data from other ear-wearable systems regarding the overpressure event.

In a forty-eighth aspect, in addition to one or more of the preceding or following aspects, or in the alternative to some aspects, the method can further include comparing a time to reflection of sound within the ear before and after the overpressure event.

In a forty-ninth aspect, in addition to one or more of the preceding or following aspects, or in the alternative to some aspects, the method can further include comparing magnitudes of signals from an outward facing microphone with magnitudes of signals from an inward facing microphone.

In a fiftieth aspect, in addition to one or more of the preceding or following aspects, or in the alternative to some aspects, the method can further include using head shadow and time of arrival effects within signals from a first ear-wearable device relative to signals from a second ear-wearable device to characterize a direction and/or magnitude of an overpressure event.

In a fifty-first aspect, in addition to one or more of the preceding or following aspects, or in the alternative to some aspects, the method can further include measuring head movement associated with the overpressure event.

In a fifty-second aspect, in addition to one or more of the preceding or following aspects, or in the alternative to some aspects, the method can further include tracking data associated with multiple overpressure events over time.

In a fifty-third aspect, in addition to one or more of the preceding or following aspects, or in the alternative to some aspects, the method can further include querying a system wearer after detection of the overpressure event.

In a fifty-fourth aspect, in addition to one or more of the preceding or following aspects, or in the alternative to some aspects, the method can further include sending data to a remote system after detection of the overpressure event.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an overpressure event herein.

FIG. 2 is a diagram of a typical pattern of rising and falling pressure associated with an overpressure event in accordance with various embodiments herein.

FIG. 3 is a schematic view of device wearer in association with an overpressure event in accordance with various embodiments herein.

FIG. 4 is a schematic view of a set of device wearers in the presence of an overpressure event in accordance with various embodiments herein.

FIG. 5 is a schematic view of an ear-wearable device in accordance with various embodiments herein.

FIG. 6 is a schematic view of an ear-wearable device within the ear of a wearer in accordance with various embodiments herein.

FIG. 7 is a schematic view of elements of gait in accordance with various embodiments herein.

FIG. 8 is a schematic view of left and right-side steps in accordance with various embodiments herein.

FIG. 9 is a schematic view of sway of an individual in accordance with various embodiments herein.

FIG. 10 is a schematic view of a query to a device wearer in accordance with various embodiments herein.

FIG. 11 is a schematic block diagram illustrating various components of an ear-wearable device in accordance with various embodiments herein.

FIG. 12 is a schematic view of components of a system in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, overpressure events can cause significant damage to structures and injury to humans and other animals. In general, the severity of these injuries depends on the proximity to the blast and the magnitude of the overpressure, with higher pressures increasing the likelihood of severe trauma or fatality. As a result, it can be important to try to classify the exposure an individual may have had to an overpressure event for clinicians to determine how best to treat them. In addition, in some cases, the results of an overpressure event injury may not manifest immediately. As such, it is desirable to monitor for potential effects on an individual after the occurrence of an overpressure event to better identify the possible injury and allow clinicians to treat the same.

Embodiments of ear-wearable devices herein can be used to identify the occurrence of overpressure events and determine various aspects of the same. By way of example, some embodiments of ear-wearable devices herein can determine a magnitude of an overpressure event. Some embodiments of ear-wearable devices herein can also determine a proximity to a blast resulting in an overpressure event. Some embodiments of ear-wearable devices herein can also determine a direction an individual was facing with respect to the cause of the overpressure event (or a direction of the event relative to the device wearer).

In addition, embodiments of systems and devices herein can monitor individuals for effects of exposure to an overpressure event. For example, embodiments of ear-wearable devices herein can monitor a device wearer for possible effects of exposure to the overpressure event including acute effects and/or chronic effects. Such effects can include those related to the vestibular system, auditory system, gastrointestinal system, respiratory system, nervous system, and the like. For example, various embodiments of ear-wearable devices herein can specifically analyze a device wearer's gait to determine possible vestibular system effects or other effects of exposure to an overpressure event.

Ear-wearable devices herein can be uniquely capable of, and valuable for, detecting overpressure events, determining parameters of the same, and/or monitoring individuals for the effects of overpressure event exposure. This is because such devices may be regularly worn by device wearers making it likely that the devices will be in-use to capture such information when an event occurs. In addition, as wearable devices that may be worn before an event ever occurs, baseline values for various parameters of an individual can be gathered allowing for a meaningful comparison of the effects of the event by comparing such values from before the event with such values after the event has occurred.

In accordance with various embodiments herein, two spatially-separated ear-wearable devices can be used (e.g., one associated with each ear) offering a number of benefits including an ability to determine a direction of the epicenter of the event and/or a distance to the epicenter of the event by using data from both ear-wearable devices and determining a difference in arrival time and/or presence of attenuation due to a head shadow effect.

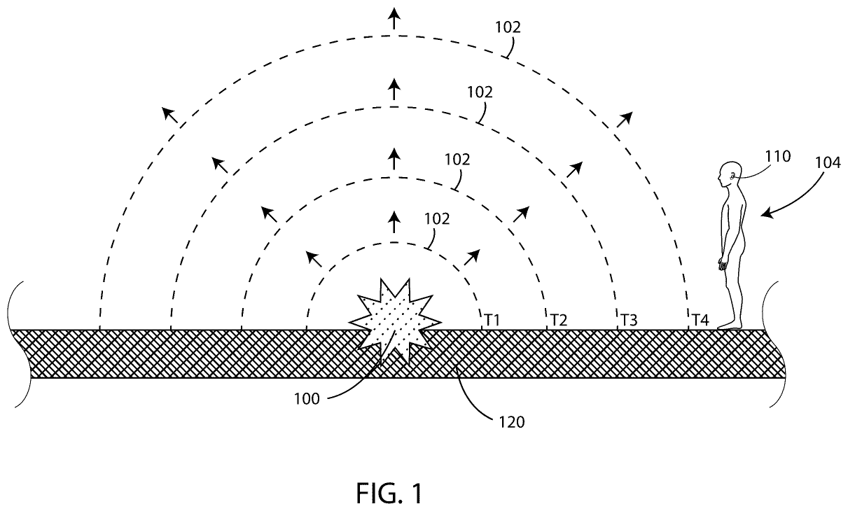

Referring now to FIG. 1, a schematic view is shown of an overpressure event herein. A blast or explosion 100 is depicted and a pressure wave 102 moves outward in all directions as depicted at times T1, T2, T3, and T4. The magnitude of the pressure wave 102 diminishes from a starting value or epicenter value as it moves outward from the epicenter. The pressure wave 102 will hit an individual 104 wearing an ear-wearable device 110 herein. In this example, the explosion 100 is depicted occurring on the ground 120. However, it will be appreciated that explosions can also occur up in the air generating a pressure wave with similar effects.

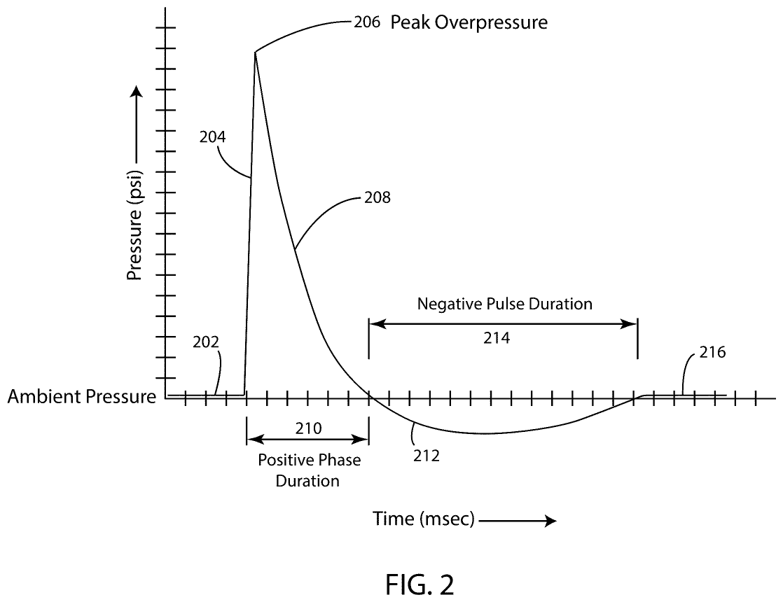

Referring now to FIG. 2, a diagram is shown of a typical pattern of rising and falling pressure associated with an overpressure event in accordance with various embodiments herein. FIG. 2 shows pressure (units of psi) versus time for a overpressure wave passing by a specific spatial point. The pressure starts at an ambient pressure value 202 then rapidly rises 204 as the pressure wave hits. The pressure wave then falls 208 and continues to fall hitting a negative pressure 212 or pressure that is lower than the starting ambient pressure 202. The total duration of time from initial rise above ambient pressure to initially falling below ambient pressure can be referred to as the positive phase duration 210. The total duration of time from when the pressure initially falls below the ambient pressure then rises back to ambient pressure 216 can be referred to as the negative pulse duration 214.

In various embodiments herein, the ear-wearable system can be configured to calculate a maximum rate of change from a starting ambient pressure to a peak pressure of the overpressure event. In various embodiments herein, the ear-wearable system can be configured to calculate a positive duration time of the overpressure event. In various embodiments herein, the ear-wearable system can be configured to calculate a negative duration time of the overpressure event.

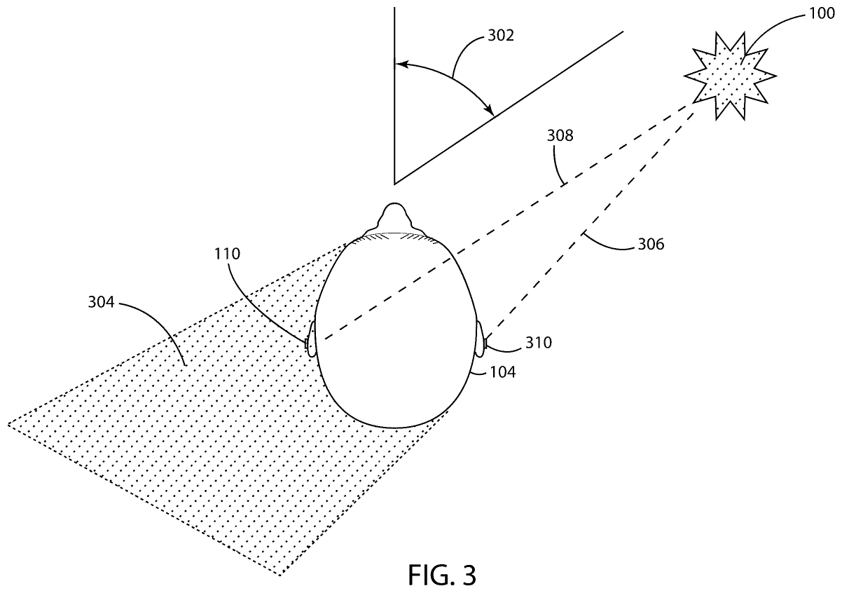

Referring now to FIG. 3, a schematic view of device wearer 104 in association with an overpressure event is shown in accordance with various embodiments herein. In this example, the device wearer 104 is wearing a first wearable device 110 herein along with a second wearable device 310. The distance between the wearable devices and the explosion 100 epicenter is different because the device wearer 104 is not directly facing the explosion 100 epicenter. In this example, the distance 306 between the explosion 100 and the second wearable device 310 is less than the distance 308 between the explosion 100 and the first wearable device 110. This difference in distance results in a difference in the time of arrival of the overpressure wave at the two wearable devices.

By evaluating the difference in the time of arrival, an estimate of the direction of the explosion (relative to a line connecting the first wearable device 110 and the second wearable device 310) can be determined. Similarly, by evaluating time of arrival, an angle 302 can be determined between a first line that is perpendicular to a line connecting the first wearable device and the second wearable device and a second line extending directly between the device wearer 104 and the explosion 100.

In addition, a distance from the device wearer to the epicenter of the overpressure event can be estimated by comparing the magnitude of the signals as gathered by the first wearable device 110 and the second wearable device 310, since the magnitude of such signals is predictably reduced the farther away from the epicenter that magnitude is measured.

FIG. 3 also illustrates a head shadow 304 effect or zone of attenuation due to the individual's head physically shielding one wearable device 110 from directly encountering the overpressure wave. In this example, first wearable device 110 is within the area of the head shadow 304 and therefore would generate signals that are attenuated relative to those gathered by second wearable device 310. In some embodiments, a degree of attenuation can be related to the angle that the device wearer is facing relative to the epicenter of the overpressure event and therefore the angle can be estimated using the same.

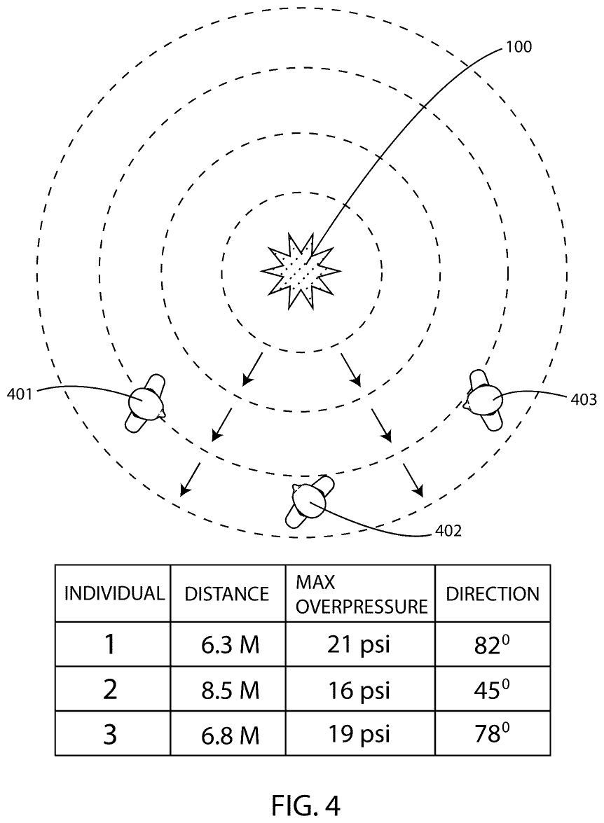

In some embodiments, systems herein can include multiple devices or sets of devices being worn by different individuals. In such a case, data generated by various devices can be exchanged with one another and/or sent to a separate system for analysis. By evaluating such data from multiple devices at multiple different positions relative to the epicenter of the overpressure event, the location and magnitude of the overpressure event can be determined more accurately. Referring now to FIG. 4, a schematic view of a set of device wearers in in the presence of an overpressure event in accordance with various embodiments herein. In this example, individual 1 (401), individual 2 (402), and individual 3 (403) are in the presence of an explosion or blast 100 creating an overpressure event. In this case, all of the individuals are wearing devices herein. As such, various aspects can be determined by evaluating data from the wearable devices including, but not limited to, an estimated distance, a maximum overpressure, and an estimated direction of the epicenter relative to the individual.

In some embodiments, locations and/or environments can be determined by the device or system and recorded along with other data. This can allow a determination of the specific locations of overpressure events. Location can be determined in various ways. In some embodiments, location can be determined using a GPS signal or a similar geolocation signal. Similarly, in some embodiments, the ear-wearable device can be configured to record identifying wireless packets (such as advertising packets) encountered and cross-reference gait against the recorded identifying wireless packets.

Identifying wireless packets can, in some cases, also be used for contact tracing and/or identifying others that may have been exposed to an overpressure event. By way of example, if the device or system detected wireless packets coming from a device that is personal to another individual, such as a smartphone, then the detection of those packets can be used as a proxy for the presence of the individual. Also, in some embodiments, the device or system herein can use sensor data, such as data from a microphone, to detect the unique signature of the voice of another individual. In various embodiments, data regarding other individuals that are present can be recorded in order to determine whether or not they were in the presence of the device wearer at the time of an overpressure event.

The term “ear-wearable device” as used herein shall refer to devices that can be used to protect a device wearer from hearing damage (e.g., hearing protection devices) and/or aid a person with normal hearing and/or aid a person with impaired hearing. Thus, term “ear-wearable device” can refer to devices that can produce optimized or processed sound for persons with normal hearing but can also include hearing assistance devices for those with hearing deficits. Ear-wearable devices herein can include sound attenuators, ear plugs, and the like. Ear-wearable devices herein can also include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices. In some embodiments, the ear-wearable device can be a hearing aid falling under 21 C.F.R. § 801.420. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the hearing assistance device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via a bone conduction pathway. In another example, the hearing assistance device can include an auditory brainstem implant, a cranial nerve (e.g., CN VIII) implant, and the like.

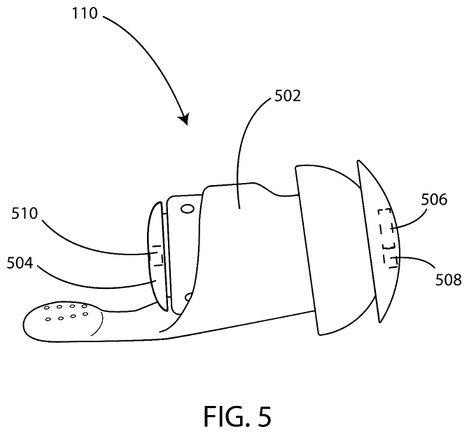

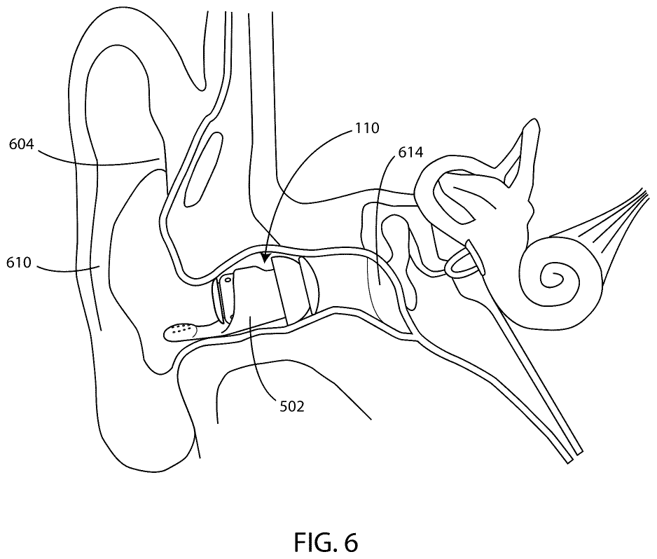

Referring now to FIG. 5, a schematic view of an ear-wearable device 110 in accordance with various embodiments herein. The ear-wearable device 110, in this example, can include a sleeve 502 and a main device housing 504 that fits within the sleeve 502. Electronic components of the ear-wearable device 110 can be disposed within the main device housing 504. In various embodiments, the ear-wearable device 110 can include an acoustic transducer or receiver or speaker 506 along with an inward facing microphone 508. The ear-wearable device 110 can also include an externally facing microphone 510. Referring now to FIG. 6, a schematic view of the ear-wearable device 110 is shown with the device fitted in the ear of a device wearer. The significant portions of the ear, in this view, include the pinna 610, ear canal 612, and tympanic membrane 614. The ear-wearable device 110 and, specifically, the sleeve 502 thereof holding the main device housing can fit within the ear canal 612 of the device wearer.

In some cases, damage from exposure to an overpressure event can cause physical damage to anatomical components of the ear. For example, in some embodiments, an overpressure event of sufficient magnitude can rupture the tympanic membrane 614 and/or create a tear in the oval window or round window. This can cause the acoustics within the ear to change in a detectable way. For example, time to reflection of sound within the ear can change based on a larger cavity after the damage to the ear. As such, in some embodiments, signals generated (and/or signal timing) by an inward facing microphone 508 of the ear-wearable device 110 can change as a result of damage to the ear. In some embodiments, the inward facing microphone 508 can be used to measure time to reflection of sound within the ear. In some embodiments, the system can be configured to compare time to reflection of sound within the ear before and after the overpressure event.

In some embodiments, the overpressure event damage causes changes to the signals generated by an inward facing microphone 508 of the ear-wearable device 110 that follow a characteristic pattern. Pattern matching techniques described herein below can be used to identify such physical damage resulting from exposure to an overpressure event.

In some embodiments, the ear-wearable system can be configured to compare magnitudes of signals from the outward facing microphone with magnitudes of signals from the inward facing microphone. In some embodiments, the ear-wearable system can provide some degree of protection to the device wearer. In some embodiments, the ear-wearable system can be configured to calculate a degree of overpressure event exposure attenuation caused by the presence of the ear-wearable device itself by comparing magnitudes of signals from the outward facing microphone with magnitudes of signals from the inward facing microphone.

### Gait Analysis

Many different characteristics of the device wearer can be sensed herein in order to detect the effects of exposure to an overpressure event. By way of example, an individual's gait may change due to effects of the overpressure event on the vestibular system (and/or other system effects). Embodiments herein can include ear-wearable devices configured to generate a set of data reflecting a gait of a device wearer after an overpressure event based on signals from at least one sensor, such as at least one of the motion sensor and the microphone. Devices and systems herein can compare the set of data against stored data reflecting a previous gait of the device wearer before exposure to an overpressure event and then characterize a status of the device wearer with respect to overpressure event exposure based on the change from the previous gait (baseline) to the current gait (post-exposure) of the device wearer.

Changes in gait can be very important in evaluating the condition of a device wearer. Such changes can reflect effects on the vestibular system suggesting that injury from an overpressure event has occurred. It can be clinically valuable to identify chronic changes that occur over a relatively long period of time (e.g., over days, weeks, months, etc.) however it may be even more critical to identify acute changes (e.g., changes occurring over a period of seconds or minutes) that may suggest injuries requiring urgent intervention.

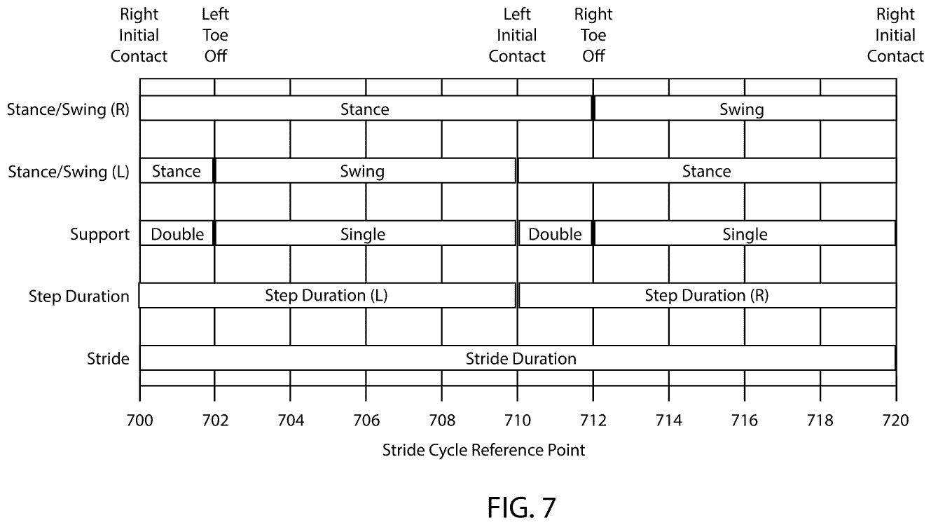

It will be appreciated that a device wearer's stride can be broken down into many different sub-elements for purposes of gait analysis herein. Referring now to FIG. 7, a diagram is shown of events occurring during strides of a device wearer for gait analysis in accordance with various embodiments herein.

At reference point 700, the right foot makes initial contact with the ground (foot fall) and both the right leg and the left leg are in a stance. Support is provided by both legs (i.e., double stance) beginning at this time. At reference point 702, the left toe leaves the ground and the left leg enters a swing while the right leg is in a stance. Support is provided by only the right leg (e.g., single) beginning at this time. At reference points 704, 706, and 708 the swing of the left leg continues. At reference point 710, the left foot makes initial contact with the ground and both the right leg and the left leg are in a stance. Support is provided by both legs beginning at this time. At reference point 712, the right toe leaves the ground and the right leg enters a swing while the left leg is in a stance. Support is provided by only the left leg (e.g., single) beginning at this time. At reference points 714, 716, and 718 the swing of the right leg continues. Reference point 720 marks the conclusion of the stride cycle whereupon if the device wearer continues to walk the cycle will repeat beginning at reference point 700.

In accordance with embodiments herein, one or more of a motion sensor and a microphone herein can detect movements and/or vibrations to identify what stage of the stride cycle the device wearer is currently in along with frequencies and time associated with the same. By way of example, reference points 700 and 710 involve the right and left feet, respectively, making initial contact with the ground. The biomechanics associated with such feet/ground contact results in characteristic acoustic and inertial changes that can be detected by one or more microphones and/or accelerometers (or other component) of a motion sensor, either alone or in combination. In some embodiments, characteristics of feet/ground contact can include a signal intensity. In some embodiments, characteristics of feet/ground contact can include a time interval. In some embodiments the spectral intensity and timing of a first, second, third, etc. microphone may be compared, summed, or subtracted to determine the spatial location of a footfall. In some embodiments, the system may determine if the footfalls are associated with the wearer or if the footfalls are associated with another individual. In some embodiments, the system may also determine if it is the left foot or the right foot making the footfall. In some embodiments, characteristics of feet/ground contact can include an angular position of one or more parts of the body. For example, as one leg swings forward (e.g., starting at reference point 702 and ending at reference point 710 for the left leg and starting at reference point 712 and ending at reference point 720 for the right leg) support by the other leg involves a characteristic vertical motion at a relatively low frequency that can be detected by a component of the motion sensor.

In some embodiments herein, a heart rate or PPG sensor can detect and/or confirm detection of footfalls. A footfall can generate a detectable signal using a heart rate or PPG sensor. In some embodiments, magnitude of motion sensor signals along with heart rate values can be used to differentiate between shuffling, typical walking, and movement due to an external force. This is because the signal from a heart rate or PPG sensor will vary depending on whether the device wearer is shuffling, exhibiting typical walking, or undergoing other movement. In some embodiments, the detection of footfalls with a motion sensor and also with a heart rate or PPG sensor can provide confirmation that the device wearer is actually wearing the ear-wearable devices as intended and not just storing them in their pockets. This is because the devices may still register footfalls with a motion sensor even if the ear-wearable device is not being worn, but a heart rate or PPG sensor associated with the device would not provide a useful signal if the device is not being worn.

Characteristic medio-lateral axis movement can also be detected by the motion sensor during different phases of the stride cycle allowing each point to be identified along with timing of the same. Left versus right steps can also be distinguished by evaluating detected medio-lateral axis movement. By way of example, a limping gait can be reflected as unequal swing durations between each leg and this type of abnormal or atypical gait can be detected by the system. As another example, a shuffling-type gait can be reflected as a measurable variability in the timing of the different phases of the stride cycle that crosses a threshold value of variability or statistics (the threshold value either being pre-selected and programmed into the device or reflecting a statistical measure of deviation from another statistical measure, e.g., an average, for the specific individual as calculated over a look-back period or during a previous calibration period or event). A shuffling-type gait or other scenarios can also be detected using acoustic information obtained from one or more microphones.

In addition, by combining the information content provided by signals associated with directional movement in the horizontal plane (as can be measured by the motion sensor, microphone, or geolocation-type sensors) with that provided by stride cycle analysis as detailed above, aspects such as step length (right, left) and stride length can be calculated. These values can also be subjected to analysis to determine various statistics, e.g., absolute values (average right step length, average left step length, average stride length) as well as ratios of the same (ratio of average right step length vs. average left step length) and measures of variability or other statistics in the same, and the like.

In some embodiments, devices and/or systems herein can be configured to determine when the wearer is stepping with a particular foot (i.e., left or right) to enable, e.g., a gait cadence that is tuned for the wearer's gait. For example, if a wearer has a “slow left foot” a device may use a motion sensor, audio information, or both, optionally with other information, to determine when a left step is occurring.

In various embodiments, the ear-wearable device can be configured to distinguish between a right step and a left step based on data from one or more sensors herein. For example, a left step can generally be detected by observing that previous movement toward the left (as part of side-to-side motion during walking) ceases coinciding with motion sensor data associated with the impact of the foot fall and/or microphone data associated with the impact of the footfall. Similarly, a right step can generally be detected by observing that previous movement toward the right ceases coinciding with motion sensor data associated with the impact of the footfall and/or microphone data associated with the impact of the footfall. In various embodiments, the ear-wearable device can be configured to distinguish between a right step and a left step based on an input received from an accessory device. By way of example, the ear-wearable device can receive a data input from another device, such as a wrist-wearable accessory device or a smart phone to distinguish between a right step and a left step. For example, if the wearer exhibits a typical walking pattern (moving left arm synced with right leg, and vice-versa), the system may receive input or determine whether a wearable device is worn on a left or right arm and use that information to determine when a left (or right) step is occurring. As another example, the device can be configured, in combination with a second device, to distinguish between right and left steps through binaural processing of motion sensor data. The motion sensor of a right ear-wearable device will provide a signal with a slightly different signature upon a right step than the motion sensor of a left ear-wearable device. Thus, the devices and/or system herein can distinguish between a right step and left step by comparing the motion sensor signatures from right and left side ear-wearable devices.

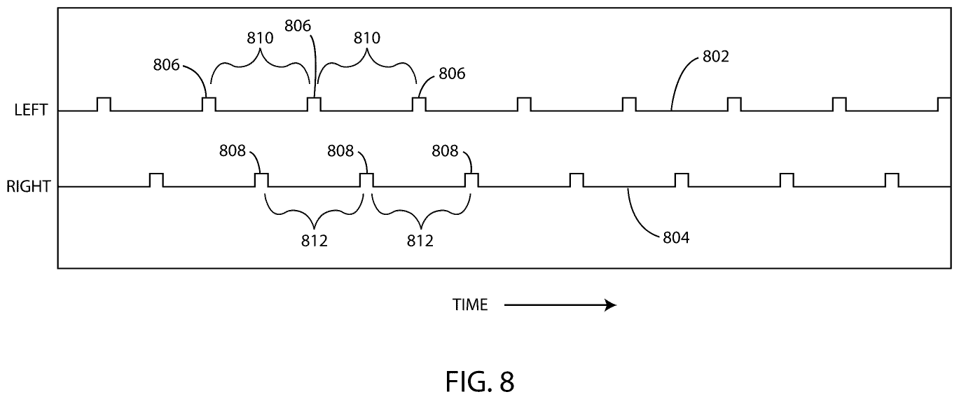

Referring now to FIG. 8, a schematic view of left and right-side steps is shown in accordance with various embodiments herein. This view shows left side gait activity 802 including left-side steps 806. The left side gait activity 802 includes a left step interval 810 between successive left-side steps 806. This view also shows right side gait activity 804 including right side step 808. The right side gait activity 804 includes a right step interval 812 between successive right-side steps 808. As previously discussed, in various embodiments the ear-wearable device can be configured to record signals from at least one sensor, such as at least one of the motion sensor and the microphone, and process the signals to characterize an existing gait of the device wearer, including left side gait activity 802 and right side gait activity 804.

In various embodiments, the ear-wearable device can be configured to generate a set of data reflecting a current gait of a device wearer based on signals from at least one sensor, such as at least one of a motion sensor and a microphone. In various embodiments, the ear-wearable device can be configured to compare a set of data (or statistics thereof) reflecting a current gait of a device wearer against stored data (or statistics thereof) reflecting a previous gait of the device wearer. The stored data reflecting a previous gait (and statistics thereof) may reflect a gait from seconds, minutes, hours, days, weeks or months in the past. In various embodiments, the ear-wearable device can be configured to characterize a health status of a device wearer based on a change from the previous gait to the current gait of the device wearer.

Changes in gait can be reflected in any of the parameters (and their related statistics) herein reflecting gait. In some embodiments, changes herein can include those of statistical significance. For example, in some embodiments, changes herein can include those reflecting a p-value of 5% or lower. In some embodiments, changes herein can include those with a 1, 2, 3, or more standard deviation difference from a previous observed value. In various embodiments, the ear-wearable device can be configured to determine whether the change from the previous gait to the current gait reflects a changing (slowing or increasing) gait tempo. In various embodiments, the ear-wearable device can be configured to determine whether the change from the previous gait to the current gait reflects a change in left/right asymmetry. In various embodiments, the ear-wearable device can be configured to determine whether the change from the previous gait to the current gait reflects a change in stride lengths.

In various embodiments, the ear-wearable device can be configured to determine whether the change from the previous gait to the current gait reflects a health status change. In various embodiments, the ear-wearable device can be configured to determine whether the change from the previous gait to the current gait reflects an elevated fall risk. An elevated fall risk may be indicated by one or more of an increase in gait asymmetry, an increase in gait timing variability, reduced gait speed, decreased stride height and length, increased front-back and side-to-side sway, increased double stance time, increased hesitancy, slowed postural transitions, wide or en bloc turns, a wide base, and out-of-plane motion, or other gait characteristics or statistics. In various embodiments, the ear-wearable device can be configured to initiate audio cues for delivery to a device wearer when an elevated fall risk is present. In some embodiments, the ear-wearable device can be configured to cease audio cues when an elevated fall risk is present and/or instruct the device wearer to pause, sit down, or use an assistive device to prevent a possibly injurious fall. In various embodiments, the ear-wearable device can be configured to send an alert to a third party when an elevated fall risk is present. In various embodiments, the ear-wearable device can be configured to send a control signal to a secondary device when an elevated fall risk is present.

In various embodiments, the ear-wearable device can be configured to cross-reference changes in gait with changes in data gathered by other sensors, such as change in activity levels of a device wearer. Activity levels can be detected based on data from various sensors including, but not limited to, motion sensor data. In some embodiments, the ear-wearable device can be configured to normalize one or more desired gait parameters based on a detected activity level as reflected in data from at least one sensor, such as a motion sensor.

In some embodiments, near-falls or stumbles can be detected through the evaluation of sensor data including, but not limited to, motion sensor and/or microphone data over a period of time and data and/or trends regarding the same can be calculated and/or reported by way of an alert or other communication to a third party such as a care provider and/or a clinician.

Characterizing the existing gait of the device wearer can be done in various ways. In some embodiments, the ear-wearable device can be configured to record signals from a sensor, such as at least one of a motion sensor and a microphone, and then process the signals to characterize an existing gait of a device wearer.

Changes in gait asymmetry can be one way of detecting injury from exposure to an overpressure event. Gait asymmetry herein can be evaluated in various ways. In some embodiments, gait asymmetry can be comparing one or more left side gait parameters with one or more corresponding right side gait parameters. As a simple example, the sound volume of left-side steps can be compared with the sound volume of right-side steps and gait asymmetry can be calculated as an average percentage difference reflecting decibels between left and right steps. As another example, the magnitude of motion sensor signals of left-side steps can be compared with the magnitude of motion sensor signals of right-side steps and gait asymmetry can be calculated as an average percentage difference (or another statistical measure) reflecting the difference between left and right steps. As another example, the step timing of left-side steps can be compared with the step timing of right-side steps and gait asymmetry can be calculated as a comparison between left and right steps. As yet another example, by evaluating motion sensor signals and, in some cases, positional or geolocation data, stride lengths can be estimated, and the estimated stride length of left-side steps can be compared with the estimated stride length of right-side steps and gait asymmetry can be calculated as a comparison between left and right stride lengths. Many other techniques of calculating a value and statistics for gait asymmetry are also contemplated herein.

In various embodiments, the ear-wearable device can be configured to operate in a first mode, wherein the first mode includes evaluating signals from at least one sensor (such as a motion sensor or a microphone or any of the other sensors described herein) to characterize a gait of a device wearer. For example, in some embodiments, the ear-wearable device can be configured to evaluate data from the motion sensor over a time period to determine a range of gait tempo values for the device wearer.

In various embodiments, the ear-wearable device, when operating in the first mode, is configured to evaluate data from the motion sensor over a time period to determine a range of gait values, such as gait tempo values, for the device wearer. The time period can be greater than or equal to 0.5, 1, 2, 4, 6, 8, 10, 12, 15, 20, 25, 30, 45, or 60 minutes or more, or an amount falling within a range between any of the foregoing. In various embodiments, the ear-wearable device can be configured to prompt a device wearer to execute specific actions while operating in the first mode.

In various embodiments, the ear-wearable device can be configured to operate in a second mode, wherein the second mode includes monitoring the device wearer for indications of overpressure event trauma and/or injury as described elsewhere herein.

In various embodiments, the ear-wearable device can be configured to match a set of data (such as data from the sensors herein) against a plurality of predetermined patterns to characterize the current gait. In various embodiments, the ear-wearable device can be configured to match the set of data against a plurality of predetermined patterns to characterize a current health status of a device wearer. In various embodiments, the ear-wearable device can be configured to determine whether the characterized current gait reflects a neurological injury and, in some cases, whether the injury or condition appears to be acute or chronic based on how suddenly the pattern has emerged in the device wearer's gait. In various embodiments, the ear-wearable device can be configured to determine whether the characterized current gait reflects a musculoskeletal injury or imbalance. In various embodiments, the ear-wearable device can be configured to alert a device wearer and/or a third party regarding a possible injury being detected. In various embodiments, the ear-wearable device can be configured to alert a device wearer and/or a third party regarding a possible injury being detected along with a recommendation to prevent further injury, such as ceasing a current activity. In some embodiments, such as where a gait pattern has been identified that matches a gait pattern associated with neurological injuries, the device can issue an alert for a coach, referee or other responsible party that an athlete (as a device wearer) may have suffered a neurological injury such as a traumatic brain injury based on the pattern of their gait. In some embodiments, such as where a gait pattern has been identified that matches a gait pattern associated with injuries, the device can issue an alert for a supervisor, military officer, or other responsible party that a soldier may have suffered an injury based on the pattern of their gait.

In various embodiments, the ear-wearable device can be configured to determine whether a change from the previous gait to the current gait reflects an injury. In various embodiments, the ear-wearable device can be configured to determine whether the change from the previous gait to the current gait reflects a neurological disease state or a neurological injury. In various embodiments, the ear-wearable device can be configured to determine whether the change from the previous gait to the current gait reflects a musculoskeletal injury.

In some embodiments, the ear-wearable device and/or system herein can also detect posture associated with gait. For example, if the device wearer is leaning too far (forward, backward, or to the side) while walking, this may negatively impact gait as well as generate an elevated fall risk. Posture can be detected using various sensors herein including, for example, an accelerometer that may be part of a motion sensor herein. In some embodiments, sensors herein can also include a gyroscope that can be used to detect angular deviations associated with gait, such as leaning forward.

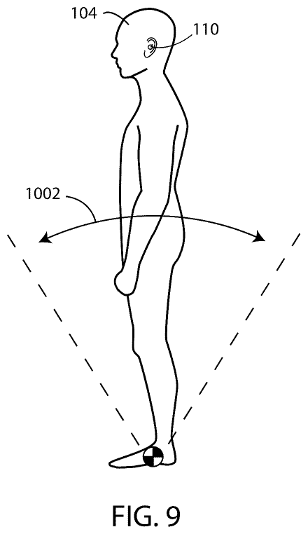

Referring now to FIG. 9, a schematic view is shown of a device wearer 104 with an ear-wearable device 110 exhibiting a degree of sway 904. Sway 904 can be measured using signals from a motion sensor. The magnitude of sway 904 can be evaluated by the ear-wearable device as derived from sensor signal thereof (such as a signal from a motion sensor, an accelerometer, a gyroscope, or the like). A change in the magnitude of sway 904 from a time before an overpressure event to a time after an overpressure event can be indicative of injury from the overpressure event.

In various embodiments herein, the ear-wearable device can be configured to characterize a current emotional status of a device wearer based on the current gait of the device wearer. For example, the ear-wearable device can be configured to match a set of data (such as data from the sensors herein) against a plurality of predetermined patterns that are associated with emotional states (angry, stressed, depressed, etc.) to characterize the current emotional status of the device wearer.

In some embodiments, a system or device herein can monitor for physiological effects of exposure to an overpressure event in the form of sleep disturbances. Sleep disturbances can be identified in various ways. For example, sleep disturbances can be identified through the evaluation of motion sensor data during normal sleeping hours. While some movement is normal during sleep, some movement patterns can be characteristic of sleep disturbances. Motion patterns gathered with sensors herein can be evaluated by matching the same against template patterns for sleep disturbances, such as using pattern matching techniques described elsewhere herein. Sleep disturbances can also be identified through the evaluation of sensor data reflecting breathing and/or breathing sounds. In some embodiments, changes in sleep disturbances for an individual from a time before exposure to an overpressure event in comparison with sleep disturbances after exposure can be indicative of the effects (such as injuries) from overpressure events.

In some embodiments, the ear-wearable system can be configured to track data associated with overpressure events over time. For example, the potential damage associated with overpressure events can be cumulative over time. As such, it can be valuable to track overpressure event exposure for an individual over time (weeks, months, years, etc.). In some embodiments herein, such data can be tracked on the ear-wearable device itself. In some embodiments, such data can be conveyed to and then tracked on a remote system, such as a remote electronic medical records system. In some embodiments, the system can be configured to generate a warning or notification when cumulative exposure has reached a predetermined level.

In some embodiments, the ear-wearable system can be configured to classify a severity of the physiological effects of the overpressure event. Such classifications can be performed in various ways. In some embodiments, classifications of severity can be based on differences in values measurable with sensors herein from a time before exposure to the overpressure event in comparison with a time after exposure to the overpressure event. In some embodiments, the classification of severity can be based on the magnitude of the change. In some embodiments, the classification of severity can be based on changes exceeding certain threshold values (which can be absolute or relative values). In some embodiments, a classification scheme herein can divide effects into a plurality of categories, such as mild, moderate, and severe effects. However, it will be appreciated that many other classification schemes are also contemplated herein.

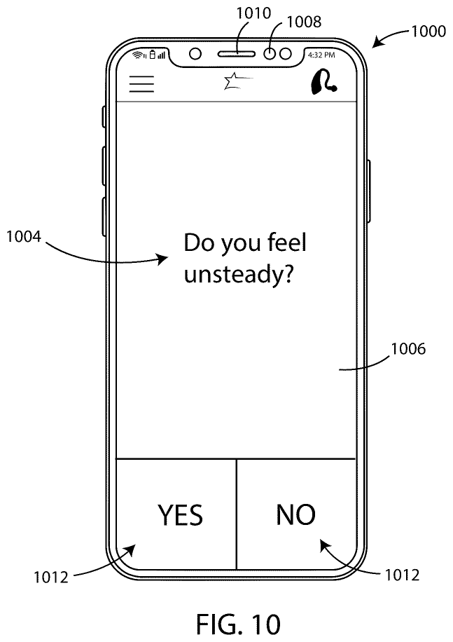

In various embodiments herein, a wearable device herein can interface with an accessory device and/or systems herein can include an accessory device. Referring now to FIG. 10, a schematic view of an accessory device 1000 is shown in accordance with various embodiments herein. The accessory device 1000 can include a display screen 1006. The accessory device 1000 can also include a speaker 1010 and a front-facing camera 1008. In some embodiments, the ear-wearable system or device can be configured to send commands to the accessory device 1000 causing the accessory device to visually display certain information and/or queries for the device wearer.

In various embodiments, a query 1004 for the device wearer can be displayed on the display screen 1006. The device wearer can be queried after detection of an overpressure event. As an example, the device wearer can be queried regarding the occurrence of the overpressure event itself and/or possible symptoms of exposure thereto. For example, a query 1004 like “Did a blast occur?” can be displayed on the display screen 1006. As another example, a query 1004 like “Do you feel unsteady?” can be displayed on the display screen 1006. The device wearer can respond by manipulating user input elements 1012, such as buttons shown on the display screen 1006. Device wearer input can then be used herein to assess possible injury from overpressure event exposure. In some embodiments, device wearer input data can be used as part of a pattern for pattern matching operations described herein. Alternatively, or in addition, queries can be presented to the device wearer audibly through speakers of the ear-wearable device itself. In addition, device wearer input data can be provided by the device wearer orally and the same can be received through one or more microphones of the system and/or devices herein.

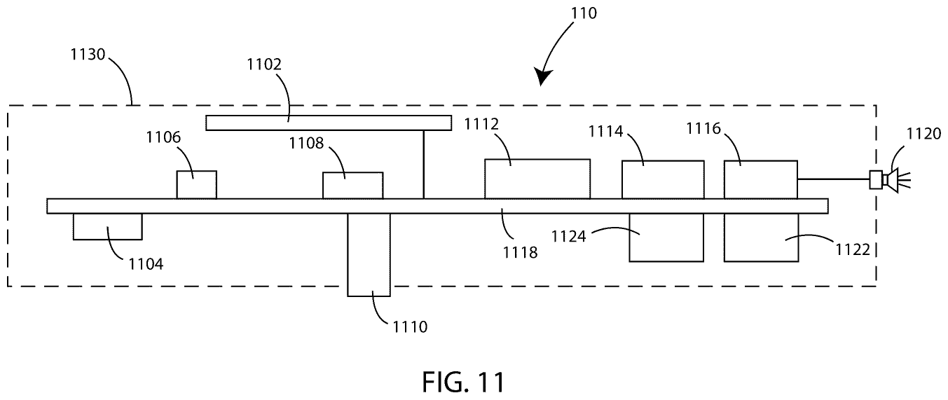

Referring now to FIG. 11, a schematic block diagram is shown illustrating various components of an ear-wearable device in accordance with various embodiments herein. It will be appreciated that many of these components can be integrated in an integrated circuit, such as with a system-on-a-chip (SOC) integration or can exist as separate components. The block diagram of FIG. 11 represents a generic ear-wearable device for purposes of illustration. However, many variations (a greater or lesser number of components) are contemplated herein. The ear-wearable device 110 shown in FIG. 11 includes several components electrically connected to a flexible mother circuit 1118 (e.g., flexible mother board) which is disposed within housing 1130. A power supply circuit 1104 can include a battery and can be electrically connected to the flexible mother circuit 1118 and provides power to the various components of the ear-wearable device 110. One or more microphones 1106 are electrically connected to the flexible mother circuit 1118, which provides electrical communication between the microphones 1106 and a digital signal processor (DSP) 1112. Among other components, the DSP 1112 incorporates or is coupled to audio signal processing circuitry configured to implement various functions described herein. A sensor package 1114 can be coupled to the DSP 1112 via the flexible mother circuit 1118. The sensor package 1114 can include one or more different specific types of sensors such as those described in greater detail below. In some cases, one or more user switches 1110 (e.g., on/off, volume, microphone directional settings) are electrically coupled to the DSP 1112 via the flexible mother circuit 1118.

An audio output device 1116 is electrically connected to the DSP 1112 via the flexible mother circuit 1118. In some embodiments, the audio output device 1116 comprises a speaker (coupled to an amplifier). In other embodiments, the audio output device 1116 comprises an amplifier coupled to a receiver 1120 adapted for positioning within an ear of a wearer. The receiver 1120 can include an electroacoustic transducer, speaker, or loudspeaker. The ear-wearable device 110 may incorporate a communication device 1108 coupled to the flexible mother circuit 1118 and to an antenna 1102 directly or indirectly via the flexible mother circuit 1118. The communication device 1108 can be a Bluetooth® transceiver, such as a BLE (Bluetooth® low energy) transceiver or other transceiver(s) (e.g., an IEEE 802.11 compliant device). The communication device 1108 can be configured to communicate with one or more external devices, such as those discussed previously, in accordance with various embodiments. In various embodiments, the communication device 1108 can be configured to communicate with an external visual display device such as a smart phone, a video display screen, a tablet, a computer, a television, a virtual or augmented reality, a hologram, or the like.

In various embodiments, the ear-wearable device 110 can also include a control circuit 1122 and a memory storage device 1124. The control circuit 1122 can be in electrical communication with other components of the device. The control circuit 1122 can execute various operations, such as those described herein. The control circuit 1122 can include various components including, but not limited to, a microprocessor, a microcontroller, an FPGA (field-programmable gate array) processing device, an ASIC (application specific integrated circuit), or the like. The memory storage device 1124 can include both volatile and non-volatile memory. The memory storage device 1124 can include ROM, RAM, flash memory, EEPROM, SSD devices, NAND chips, and the like. The memory storage device 1124 can be used to store data from sensors as described herein and/or processed data generated using data from sensors as described herein.

In various embodiments, a spatial location determining circuit (or geolocation circuit) can be included and can take the form of an integrated circuit that can include components for receiving signals from GPS, GLONASS, BeiDou, Galileo, SBAS, WLAN, BT, FM, and/or NFC type protocols.

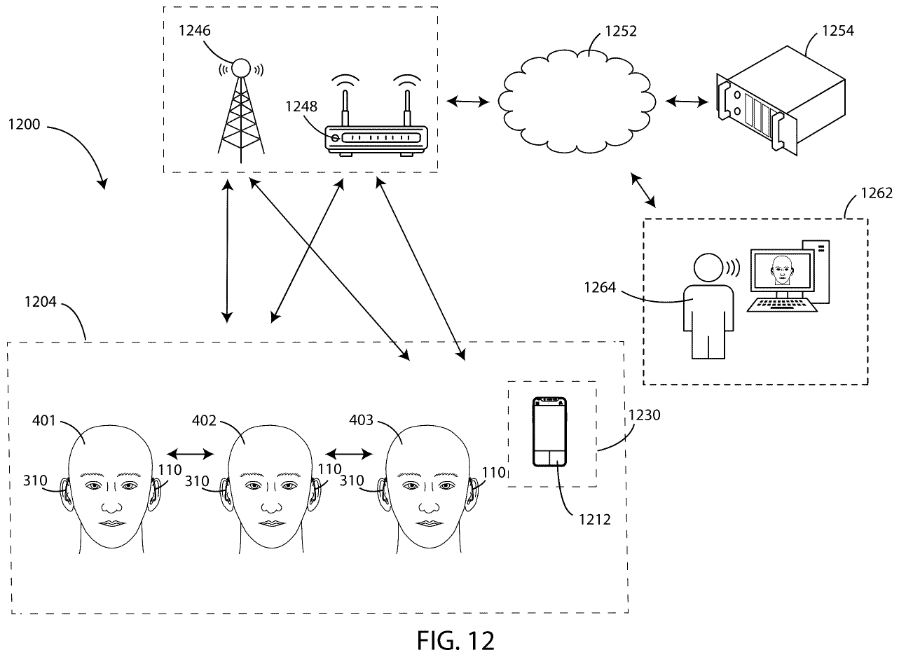

Referring now to FIG. 12, a schematic view is shown of components of a system 1200 in accordance with various embodiments herein. FIG. 12 shows a first device wearer 401, a second device wearer 402, and a third device wearer 403, each with a first ear-wearable device 110 and a second ear-wearable device 310. The device wearers are at a first location or field location 1204. The system can include and/or can interface with other devices 1230 at the first location 1204. The other devices 1230 in this example can include an accessory device 1212, which could be a smart phone or similar mobile communication/computing device in some embodiments. In some embodiments, the accessory device 1212 can be used as a gateway to convey communications or data to a remote system or location. In some embodiments herein, communications can be conveyed between wearable devices peer-to-peer, such that the devices of the wearers can exchange data between themselves, such as data on overpressure events.

FIG. 12 also shows communication equipment including a cell tower 1246 and a network router 1248. FIG. 12 also schematically depicts the cloud 1252 or a similar data communication network. FIG. 12 also depicts a cloud computing resource 1254. The communication equipment can provide data communication capabilities between the ear-wearable devices 110, 310 and other components of the system and/or components such as the cloud 1252 and cloud resources such as a cloud computing resource 1254. In some embodiments, the cloud 1252 and/or resources thereof can host an electronic medical records system. In some embodiments, the cloud 1252 can provide a link to an electronic medical records system.

FIG. 12 also shows a remote location 1262. The remote location 1262 can be the site of a third party 1264, which can be a medical professional, an officer, a commander, a care provider, a team leader, or the like. The third party 1264 can receive reports regarding the overpressure events and/or effects on the device wearer of the same.

Specifically, in various embodiments, the ear-wearable devices can be configured to send an alert to a third party 1264 if a possible overpressure event is detected. In some embodiments, the third party 964 can provide instructions for the device wearer regarding actions to take. In various embodiments, the system 1200 can be configured to send information regarding an overpressure event and/or the effects thereof to an electronic medical record system.

### Voice/Speech Monitoring

In various embodiments, monitoring a device wearer for the effects of exposure to an overpressure event can include monitoring for changes in voice and/or speech of the device wearer. Such changes can include changes in speech parameters including, but not limited to, the quantity of speech over a given time frame, the clarity or slurring of speech, maximum phonation time, maximum duration of breath phrases, maximum sound pressure level, maximum voice area in voice-range profiles, voice handicap index (VHI) values, and the like. In some cases, such changes can be used as a proxy for cognitive performance effects.

In various embodiments, the device can be configured to detect the device wearer's own voice (as opposed to a third party voice) in order to provide a better proxy for cognitive exertion of the device wearer. Own voice detection can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer. In some embodiments, such as where there are two or more ear-worn devices, own-voice detection can be performed and/or enhanced through correlation or matching of intensity levels and or timing.

In some cases, the system can include a bone conduction microphone to preferentially pick up the voice of the device wearer. In some cases, the system can include a directional microphone that is configured to preferentially pick up the voice of the device wearer. In some cases, the system can include an intracanal microphone (a microphone configured to be disposed within the ear-canal of the device wearer) to preferentially pick up the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, the system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F0. A voice detection circuit is adapted to receive the second output signal B and the pitch output F0 and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

### Methods

Many different methods are contemplated herein, including, but not limited to, a method of detecting an overpressure event, methods of monitoring a device wearer for effects of exposure to an overpressure event, methods of detecting an overpressure event injury, and the like.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In various embodiments, a method detecting an overpressure event can include evaluating signals from one or more sensors (including, but not limited to, a motion sensor, an audio input, a microphone, a pressure sensor, and the like) to detect a pressure wave exceeding a threshold magnitude. In some embodiments, the threshold magnitude can be about 0.1, 0.25, 0.5, 1, 2, 3, 4, 5, 7.5, 10, 12.5, 15, 17.5, 20, 25, or 30 psi or higher, or an amount falling within a range between any of the foregoing.

In various embodiments, a method detecting an overpressure event can include evaluating signals from one or more sensors to detect a pressure wave exceeding a threshold rate of change. For example, a threshold rate of change can be an increase in pressure of at least 1, 2, 3, 4, or 5 psi in 500, 200, 300, 200, 100 or 50 milliseconds or less.

In various embodiments, a method detecting an overpressure event can include evaluating signals from one or more sensors to detect a pressure wave exceeding a threshold rate of change and with a peak pressure exceeding a threshold magnitude.

In an embodiment, a method of evaluating overpressure event injury is included. The method can include detecting an overpressure event using one or more sensors of a sensor package of an ear-wearable device and monitoring physiological effects of the overpressure event using one or more sensors of the sensor package.

In an embodiment, the method can further include monitoring the physiological effects by comparing baseline values versus after overpressure event values. In an embodiment, the method can further include monitoring the physiological effects by comparing baseline values versus after overpressure event values for one or more of balance, gait, posture, blood pressure, heart rate and blood perfusion.

In an embodiment, the method can further include determining a direction of the overpressure event source relative to a system wearer. In an embodiment, the method can further include determining a direction of the overpressure event source relative to multiple system wearers.

In an embodiment, the method can further include determining a magnitude of the overpressure event. In an embodiment, the method can further include estimating a magnitude of the overpressure event that saturates one or more sensors of the sensor package. In an embodiment, the method can further include calculating a maximum rate of change from a starting ambient pressure to a peak pressure of the overpressure event.

In an embodiment, the method can further include calculating a positive duration time of the overpressure event. In an embodiment, the method can further include calculating a negative duration time of the overpressure event.

In an embodiment, the method can further include classifying a severity of the physiological effects of the overpressure event.

In an embodiment, the method can further include receiving data from other ear-wearable systems regarding the overpressure event.

In an embodiment, the method can further include comparing a time to reflection of sound within the ear before and after the overpressure event.

In an embodiment, the method can further include comparing magnitudes of signals from an outward facing microphone with magnitudes of signals from an inward facing microphone.

In an embodiment, the method can further include using head shadow and time of arrival effects within signals from a first ear-wearable device relative to signals from a second ear-wearable device to characterize a direction and/or magnitude of an overpressure event.

In an embodiment, the method can further include measuring head movement associated with the overpressure event.

In an embodiment, the method can further include tracking data associated with multiple overpressure events over time.

In an embodiment, the method can further include querying a system wearer after detection of the overpressure event.

In an embodiment, the method can further include sending data to a remote system after detection of the overpressure event.

In various embodiments herein, methods can include one or more operations of calculating one or more gait parameters, recording signals from at least one of the motion sensor and the microphone and processing the signals to characterize a gait of the device wearer, normalizing one or more gait parameters based on a detected activity level as reflected in data from the motion sensor, evaluating data from the motion sensor over a time period to determine a range or statistics relating to gait tempo values for the device wearer, distinguishing between a right step and a left step based on an input received from an accessory device, characterizing a gait of the device wearer at varying levels of physical exertion, characterizing a gait of the device wearer at varying levels of physical exertion, recording identifying wireless packets encountered and cross-reference gait against the recorded identifying wireless packets, generating a set of data reflecting a current gait of a device wearer based on signals from at least one of the motion sensor and the microphone, matching the set of data against a plurality of predetermined patterns to characterize the current gait, matching the set of data against a plurality of predetermined patterns to characterize an overpressure event injury status of the device wearer, generating a set of data reflecting a current gait of a device wearer based on signals from at least one of the motion sensor and the microphone, comparing the set of data against stored data reflecting a previous gait of the device wearer, and/or characterizing a health status or injury status of the device wearer based on a change from the previous gait to the current gait of the device wearer.

Aspects of system/device operation described elsewhere herein can be performed as operations of one or more methods in accordance with various embodiments herein.

### Pattern Identification and Matching

It will be appreciated that in various embodiments herein, a device or a system can be used to detect an overpressure event injury status or condition, a neurological injury or condition, a musculoskeletal injury or condition, a gait pattern or patterns indicative of a type of gait, or the like. Such patterns can be detected in various ways. Some techniques are described elsewhere herein, but some further examples will now be described.

As merely one example, one or more sensors can be operatively connected to a controller (such as the control circuit described in FIG. 11) or another processing resource (such as a processor of another device or a processing resource in the cloud).

The controller or other processing resource can be adapted to receive data representative of the device wearer, such as data representative of a gait of the device wearer from one or more of the sensors and/or determine gait statistics of the subject over a monitoring time period based upon the data received from the sensor(s). As used herein, the term “data” can include a single datum or a plurality of data values or statistics. The term “statistics” can include any appropriate mathematical calculation or metric relative to data interpretation, e.g., probability, confidence interval, distribution, range, or the like.

Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of an occurrence of a particular health status/event, injury or condition (positive example patterns), one or more predetermined gait patterns that serve as patterns indicative of the absence of a particular health status/event, injury or condition (negative example patterns), or the like. As merely one scenario, if a pattern is detected in an individual that exhibits similarity crossing a threshold value to a particular positive example pattern or substantial similarity to that pattern, wherein the pattern is specific for a particular health status/event, injury or condition, then that can be taken as an indication of an occurrence of a particular health status/event, injury or condition.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with an individual over the monitoring time period can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable devices and/or systems herein can be configured to periodically update the machine learning classification model based on data regarding the device wearer.

In some embodiments, a training set of data can be used in order to generate a machine learning classification model. The input data can include microphone and/or sensor data as described herein as tagged/labeled with binary and/or non-binary classifications of overpressure injury. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of gait) can include k-nearest neighbors, decision trees, naive Bayes approaches, random forest approaches, and gradient boosting approaches amongst others.

In various embodiments, the device and/or system herein is configured to execute operations to generate or update the machine learning model on the ear-wearable device itself. In some embodiments, the ear-wearable device may convey data to another device such as an accessory device or a cloud computing resource in order to execute operations to generate or update a machine learning model herein.

In various embodiments herein, threshold values used herein (as described at various points herein) can be calculated or otherwise derived through analysis of data regarding the device wearer. For example, in some embodiments, a threshold value can be set through evaluation of previous events related to the health status of the device wearer. In some cases, such events can be detected by the ear-wearable device(s). In other cases, such events can be provided as input to the ear-wearable device(s) from another system, device, or third party. In some embodiments, the threshold value can be related to the occurrence of such events. In some embodiments, the threshold value can be related to the prediction of the occurrence of such events based on a comparison of past data associated with the occurrence of such events and current data. In some embodiments, the threshold value can be related to a characterization of the device wearer's gait associated with the occurrence of such events. In some embodiments, the threshold value can divide categories of relevance for overpressure event injury such that a process of categorization also calculates threshold value(s). Categorization and/or calculation of threshold values can, in some cases, be performed using a machine learning approach including for example, an unsupervised machine learning approach. However, in some scenarios, supervised machine learning approaches can also be used. In some embodiments, calculation of threshold values can be performed using statistical approaches.

### Sensor Package

Various embodiments herein include one or more sensors. Specifically, devices and systems herein can include one or more sensors (including one or more discrete or integrated sensors) to provide data for use with operations to evaluate and/or characterize the gait of a device wearer. Further details about the sensors are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG (photoplethysmography) sensor data.

In various embodiments, the gait of the device wearer is characterized using data produced by at least one of the motion sensor and the microphone. In various embodiments, other sensors can also be included such as at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (such as a core body temperature sensor, skin temperature sensor, ear-canal temperature sensor, or another temperature sensor), a motion sensor, an electroencephalograph (EEG) sensor, and a respiratory sensor. In various embodiments, the motion sensor can include at least one of an accelerometer and a gyroscope.

Devices herein can specifically include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motion sensors can be used to track movements of a patient in accordance with various embodiments herein.

In some embodiments, the motion sensors can be disposed in a fixed position with respect to the head of a patient, such as worn on or near the head or ears. In some embodiments, operatively connected motion sensors can be worn on or near another part of the body such as on a wrist, arm, or leg of the patient.

According to various embodiments, sensors herein can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS) circuit, a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (or electromyography-EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, sensors herein can be part of an ear-wearable device. However, in some embodiments, the sensors utilized can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) herein can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

In some embodiments, the sensor package can include a sound input device. The sound input device can generate an electric signal based on pressure waves. In some embodiments, the sound input device can specifically include a microphone. As used herein, the term “microphone” shall include reference to all types of devices used to capture sounds including various types of microphones (including, but not limited to, carbon microphones, fiber optic microphones, dynamic microphones, electret microphones, ribbon microphones, laser microphones, condenser microphones, cardioid microphones, crystal microphones) and vibration sensors (including, but not limited to accelerometers and various types of pressure sensors). Microphones herein can include analog and digital microphones. Systems herein can also include various signal processing chips and components such as analog-to-digital converters and digital-to-analog converters. Systems herein can operate with audio data that is gathered, transmitted, and/or processed reflecting various sampling rates. By way of example, sampling rates used herein can include 8,000 Hz, 11,025 Hz, 16,000 Hz, 22,050 Hz, 32,000 Hz, 37,800 Hz, 44,056 Hz, 44,100 Hz, 47,250 Hz, 48,000 Hz, 50,000 Hz, 50,400 Hz, 64,000 Hz, 88,200 Hz, 96,000 Hz, 176,400 Hz, 192,000 Hz, or higher or lower, or within a range falling between any of the foregoing. Audio data herein can reflect various bit depths including, but not limited to 8, 16, and 24-bit depth. Microphones herein can include both directional and omnidirectional microphones. In some embodiments, microphones herein can be configured to be sensitive to sounds coming from the direction of the device wearer's feet to more sensitively pick up the sound of foot falls while walking. In some embodiments, microphones herein can include inward facing microphones to be more sensitive to pickup foot fall sounds through the body.

In some embodiments herein, one or more microphones (externally facing, inward facing, etc.) can be a microphone with a high saturation limit (e.g., the maximum pressure the microphone can generate a signal for). By way of example, in some embodiments the microphone can be a MEMS or electret microphone.

In some embodiments, an ear-wearable system can be configured to estimate a magnitude of the overpressure event that saturates one or more sensors of the sensor package. For example, where a microphone hits a saturation point, then data from another sensor such as a motion sensor can be used to estimate the magnitude of the overpressure event in combination with the microphone data. For example, substantial overpressure events can result in a degree of head movement and/or device movement. The degree of head movement can be related to a magnitude of an overpressure event over a saturation limit of a microphone or another sensor. As such, in some embodiments, the ear-wearable system can be configured to measure head movement associated with the overpressure event. In this way, data from a non-saturating sensor can be used to estimate magnitudes of overpressure events that are quite high.

An eye movement sensor herein may be, for example, an electrooculographic (EOG) sensor, such as an EOG sensor disclosed in commonly owned U.S. Pat. No. 9,167,356, which is incorporated herein by reference.

A pressure sensor herein can be, for example, a MEMS-based pressure sensor, a piezo-resistive pressure sensor, a flexion sensor, a strain sensor, a diaphragm-type sensor and the like. Pressure sensors herein can include those capable of measuring pressures associated with an overpressure event. By way of example, pressure sensors herein can measure pressures of 0.1, 0.25, 0.5, 1, 2, 3, 4, 5, 7.5, 10, 12.5, 15, 17.5, 20, 25, or 30 psi or higher, or an amount falling within a range between any of the foregoing.

A temperature sensor herein can be, for example, a thermistor (thermally sensitive resistor), a resistance temperature detector, a thermocouple, a semiconductor-based sensor, an infrared sensor, or the like.

A blood pressure sensor herein can be, for example, a pressure sensor. The heart rate sensor can be, for example, an electrical signal sensor, an acoustic sensor, a pressure sensor, an infrared sensor, an optical sensor, or the like.

An oxygen saturation sensor (such as a blood oximetry sensor) herein can be, for example, an optical sensor, an infrared sensor, a visible light sensor, or the like.

An electrical signal sensor herein can include two or more electrodes and can include circuitry to sense and record electrical signals including sensed electrical potentials and the magnitude thereof (according to Ohm's law where V=IR) as well as measure impedance from an applied electrical potential.

It will be appreciated that sensors herein can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of a medical device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable system comprising:
a control circuit; and
a sensor package, wherein the sensor package is in electrical communication with the control circuit, the sensor package comprising
a motion sensor; and
one or more sound input devices;
wherein the ear-wearable system is configured to
detect an overpressure event using the sensor package; and
monitor physiological effects of the overpressure event.

2. The ear-wearable system of claim 1, the physiological effects comprising vestibular system effects.

3. The ear-wearable system of claim 1, the physiological effects comprising at least one selected from the group consisting of a change in balance, a change in gait, a change in posture, a change in blood pressure, and a change in heart rate.

4. (canceled)

5. The ear-wearable system of claim 1, the physiological effects comprising speech changes.

6-7. (canceled)

8. The ear-wearable system of claim 1, wherein the ear-wearable system is configured to monitor the physiological effects by comparing baseline values versus after overpressure event values for one or more of balance, gait, posture, blood pressure, heart rate and blood perfusion.

9. (canceled)

10. The ear-wearable system of claim 1, the overpressure event comprising a peak overpressure of greater than 5 psi.

11. (canceled)

12. The ear-wearable system of claim 1, wherein the ear-wearable system is configured to determine a direction of the overpressure event source relative to a system wearer.

13. (canceled)

14. The ear-wearable system of claim 1, wherein the ear-wearable system is configured to determine a magnitude of the overpressure event.

15. The ear-wearable system of claim 1, wherein the ear-wearable system is configured to estimate a magnitude of the overpressure event that saturates one or more sensors of the sensor package.

16. The ear-wearable system of claim 1, wherein the ear-wearable system is configured to calculate a maximum rate of change from a starting ambient pressure to a peak pressure of the overpressure event.

17-21. (canceled)

22. The ear-wearable system of claim 1, wherein the one or more sound input devices includes an inward facing microphone.

23. The ear-wearable system of claim 22, wherein the inward facing microphone is used to measure a time to reflection of sound within the ear.

24. The ear-wearable system of claim 23, wherein the ear-wearable system is configured to compare a time to reflection of sound within the ear before and after the overpressure event.

25. The ear-wearable system of claim 1, the sensor package further comprising:
an outward facing microphone; and
an inward facing microphone; and
wherein the ear-wearable system is configured to compare magnitudes of signals from the outward facing microphone with magnitudes of signals from the inward facing microphone.

26. The ear-wearable system of claim 1, further comprising:
a first ear-wearable device; and
a second ear-wearable device, wherein each of the first ear-wearable device and the second ear-wearable device has a separate control circuit and sensor package.

27. The ear-wearable system of claim 26, wherein data from sensors of both the first ear-wearable device and the second ear-wearable device are used to detect and/or analyze the overpressure event.

28. The ear-wearable system of claim 27, wherein the ear-wearable system is configured to use head shadow and time of arrival effects within signals from the first ear-wearable device relative to signals from the second ear-wearable device to characterize a direction and/or magnitude of the overpressure event.

29. The ear-wearable system of claim 1, wherein the ear-wearable system is configured to measure head movement associated with the overpressure event.

30-31. (canceled)

32. The ear-wearable system of claim 1, wherein the ear-wearable system is configured to track data associated with multiple overpressure events over time.

33. The ear-wearable system of claim 1, wherein the ear-wearable system is configured to query a system wearer after detection of the overpressure event.

34-54. (canceled)
